(19) United States
(12) Patent Application Publication
(10) Pub. No.: US 2025/0248748 A1
(43) Pub. Date: Aug. 7, 2025

(54) TELESCOPIC PLATE

(71) Applicant: TST RAKOR VE TIBBI ALETLER SANAYI VE TICARET LIMITED SIRKETI, Pendik - Istanbul (TR)

(72) Inventors: Muharrem Inan, Kartal Istanbul (TR); Ahmet Fethi Polat, Pendik - Istanbul (TR)

(21) Appl. No.: 18/855,634

(22) PCT Filed: Apr. 5, 2023
(86) PCT No.: PCT/TR2023/050321

§ 371 Date: Oct. 9, 2024

(30) Foreign Application Priority Data
Apr. 12, 2022 (TR) 2022/005837

(51) Int. Cl. A61B 17/80; A61B 17/00

(52) U.S. Cl. CPC A61B 17/8023; A61B 2017/00991

(57) ABSTRACT

The invention relates to a telescopic plate (8) designed to prevent or stabilize fractures and correct long (femur) bone deformities in children older than 18 months with osteogenesis imperfecta (glass bone).

### TECHNICAL FIELD

The present invention relates to plates used in long (femur) bone fractures in children over 18 months old with osteogenesis imperfecta (glass bone).

In particular, the invention relates to a telescopic plate designed to prevent or stabilize long (femur) bone fractures and correct long (femur) bone deformities in children older than 18 months with osteogenesis imperfecta (glass bone).

### PRIOR ART

The femur or thigh bone is the longest, bulkiest and strongest bone in the human body. It forms part of the hip and knee. It is the long bone that comes just after the hip.

The diaphysis is the main part, right at central part of the long bones. The diaphysis is made of cortical bone and contains bone marrow and adipose tissue. It contains both red and yellow bone marrow. The main ossification takes place in the diaphysis.

Bone fractures can occur in young children during the bone development period. In this case, stabilization of the long bone (femur) fracture is required especially for the healing of the long bone (femur) fracture. The bone needs to be stabilized without hindering its growth.

Telescopic intramedullary nailing with external fixation is indicated for long bone (femur) fracture stabilization, correction of long bone deformity, and limb lengthening in young children.

In a paediatric patient to whom nailing or fixator technique is applied, while the development continues, the nail or fixator also elongates parallel to the growth. In this way, the child's development is not hindered.

The nail is inserted into the bone in the medulla canal. Since it is placed into the bone with little incision, the risk of infection is low.

The fixator is placed outside the skin. Therefore, the comfort of life is partially reduced. Since there are few incisions, the risk of infection is low.

Both techniques are used together when the mechanical strength of the nail needs to be supported. The mechanical strength of the nail is increased with the fixator; however, it also limits the patient's comfort of life.

While extensible nailing and fixation techniques are used in current treatments, both techniques are used together in some cases.

For example, it is used in cases where nail strength is insufficient in telescopic nailing technique, in cases where thicker nails are required but cannot be used, and to prevent deformity or fracture during bone development/elongation. Since the fixators used are on the skin, irritation and infections may occur on the skin.

Application no TR 2014/12961 describes the high tibial osteotomy external fixator. However, it causes the problems described above and negatively affects the comfort of life.

In the application no TR 2015/05232, the telescopic nail is described. However, nails alone are not enough. In cases where the nail strength is insufficient, an external fixator is used.

There is a need for new technical developments that can be placed under the skin on the bone surface, can be used alone in the treatment or can be used with the nailing technique, increasing the mechanical strength of the nail and affecting the comfort of life.

### The Aims of the Invention

Based on the state of the art, the aim of the invention is to develop a telescopic plate used in long bone (femur) fractures in children older than 18 months with osteogenesis imperfecta (glass bone) disease, where the problems in existing structures are eliminated.

Another aim of the invention is to extend the telescopic plate in long bone (femur) fractures and to prevent the elongation of the telescopic plate when desired.

Another aim of the invention is to form the proximal and distal region plates on the telescopic plate in such a way that the form passing into the middle body of the plate prevents rotation.

Another aim of the invention is to develop a telescopic plate with a sliding (telescopic) structure, which provides fracture healing by being mounted on the bone, so as not to hinder the growth of the paediatric patient.

Another aim of the invention is to develop a telescopic plate designed for the treatment of children with osteogenesis imperfecta (glass bone) disease that can be applied with or without nails.

Another aim of the invention is to develop a telescopic plate that can be used instead of the fixators used in the state of the art, has a low-profile design, can be extended by moving within each other and has a locking feature at the desired length.

Another aim of the invention is to develop a telescopic plate that is fixed to the long bone (femur) with the help of screws by placing it under the skin in long bone (femur) fractures for the treatment of children with osteogenesis imperfecta (glass bone) disease.

Another object of the invention is to develop a telescopic plate that can stabilize different fracture sites on the long (femur) bone.

Another aim of the invention is to prevent infections that will occur later by placing it in the long (femur) bone under the skin laterally with a surgical operation and to prevent ergonomic discomfort caused by fixators.

In order to achieve the aforementioned goals, a telescopic plate has been developed that provides important technical advantages, performs fracture treatment without hindering the bone development of children, and is used in long bone (femur) fractures in children older than 18 months with osteogenesis imperfecta (glass bone) disease.

### DESCRIPTION OF DRAWINGS

FIG. 1; is a disassembled drawing of a telescopic plate in a representative embodiment of the invention.

FIG. 2; is the drawing of the telescopic plate assembly view in a representative embodiment of the invention.

FIG. 3; is the drawing of the telescopic plate placed on the bone in a representative embodiment of the invention.

FIG. 4; is the drawing of the elongated view of the telescopic plate in the proximal and distal directions as a result of the growth of the bone in a representative embodiment of the invention.

FIG. 5; is the drawing of the assembled and disassembled telescopic plate elements In a representative embodiment of the invention.

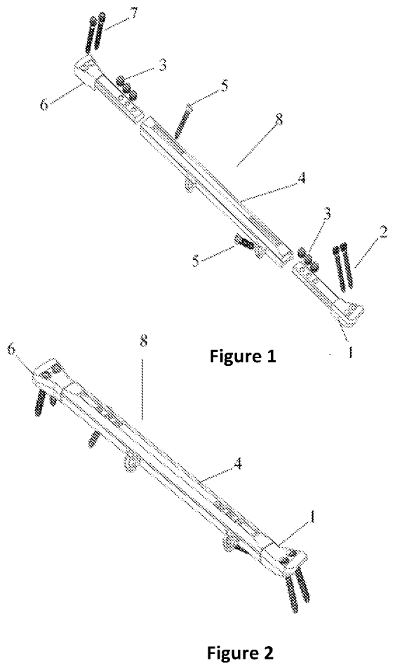

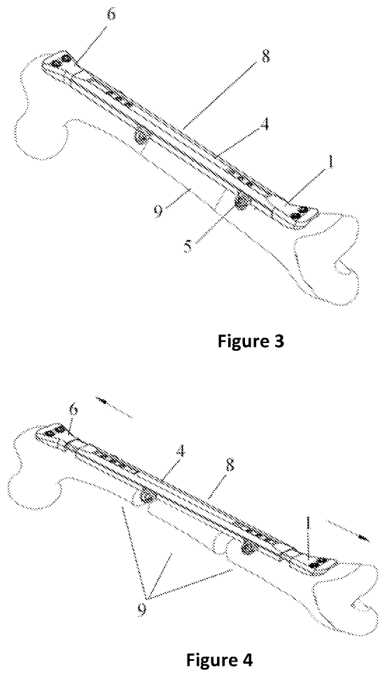

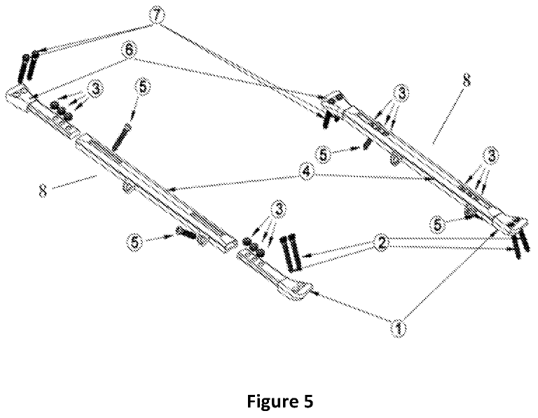

### REFERENCE NUMBERS

| | |
|---|---|
| 1 | Distal region plate |
| 2 | Distal region lock screws |
| 3 | Fixing screws |
| 4 | Plate central body |
| 5 | Diaphysis lock screws |
| 6 | Proximal region plate |
| 7 | Proximal region lock screws |
| 8 | Telescopic plate |
| 9 | Bone |

### DETAILED DESCRIPTION OF THE INVENTION

the telescopic plate (8) design, which has a low-profile design, can move within each other and has a locking feature at the desired length, has been developed in order to be used instead of the fixators used in the state of the art.

Telescopic plate (8) is surgically placed under the skin from the bone (9) lateral to prevent infections that will occur later, and ergonomic discomforts caused by fixators are prevented.

The required stability is provided in the distal, proximal, and cortical regions with locking and unlocking screws. It is designed with a thin profile suitable for the distal and proximal anatomy of the long (femur) bone (9) and can be designed in different lengths. These features facilitate the stability of different fracture sites on the long (femur) bone (9).

Telescopic plate (8) is a plate designed for the treatment of children with osteogenesis imperfecta (glass bone) disease, which can be applied with or without nails. The telescopic plate (8) is placed under the skin and fixed to the bone (9) with the help of screws.

The telescopic plate (8) design is designed in a telescopic structure that does not interfere with the growth of the paediatric patient. In desired cases, interlocking can be prevented with the fixing screws (3). The elongation of the telescopic plate (8) and the prevention of elongation in desired situations are the biggest innovations of the telescopic plate (8).

The telescopic plate (8), consists of six parts. The distal region plate (1) is fixed to the distal of the femur bone (9) with the distal region lock screws (2).

The orientations of the distal region lock screws (2) have been determined so that they do not come to the child's growth line. The distal region plate (1) and the proximal region plates (6) move by sliding within the central body (4). By means of this sliding movement, the elongation of the telescopic plate (8) takes place along with the growth.

The fixing screws (3) are tightened with the help of the threads on the distal and proximal plates (1,6), and after tightening the plate compresses the central body (4).

By means of this compression, the intertwined sliding movement of the telescopic plate (8) is prevented. The telescopic plate (8) is fixed in desired situations and does not move. The proximal region plate (6) is fixed with proximal region lock screws (7). The plate central body (4) is fixed to the bone (9) with diaphysis lock screws (5).

The form of the proximal and distal region plates (6, 1) passing to the plate central body (4) is designed to prevent rotation. Since there is no rotation, the telescopic plate (8) moves axially in the proximal and distal directions.

The invention relates to plates used to prevent or stabilize long (femur) bone fractures and correct long (femur) bone deformities in children older than 18 months with osteogenesis imperfecta (glass bone) disease, comprising The distal region plate (1) and the proximal region plate (6), which form the telescopic plate (8), which can be extended by moving within each other and locked at the desired length, at least one plate middle body (4) in which the distal region plate (1) and the proximal region plate (6) move in a telescopic structure.

It comprises the distal region plate (1), which is fixed to the distal of the femur bone (9) with the distal region lock screws (2), which are formed so as not to come to the child's growth line. Proximal to the femur bone (9), the proximal region comprises the proximal region plate (6), which is fixed with proximal region lock screws (7).

It comprises the plate central body (4), which is between the distal region plate (1) and the proximal region plates (6), distal region plate (1) and proximal region plates (6), which slide in the plate central body (4), allowing the telescopic plate (8) to extend in the distal and proximal directions.

It comprises fixing screws (3) that are tightened with the help of the threads on the distal and proximal region plates (1, 6) and that prevent the telescopic plate (8) from making any sliding motion by tightening the plate central body (4) after tightening. It comprises the plate central body (4), which is fixed to the bone (9) with diaphysis lock screws (5).

In order for the telescopic plate (8) to move axially in the proximal and distal directions, the proximal and distal region plates (6, 1), which are formed to prevent rotation, have a form that passes into the plate central body (4).

Inside the distal region plate (1) and the proximal region plate (6), which are designed with a thin profile and suitable for the distal and proximal anatomy of the femur bone (9), fixed with locking and unlocking screws in the distal, proximal and cortical regions of the femur bone (9), it comprises the plate central body (4), on which it moves in a telescopic structure.

It comprises the distal region plate (1), the proximal region plate (6), and the plate central body (4) in which stability is achieved with locking and unlocking screws in distal, proximal and cortical regions, and which has a profile structure suitable for the distal and proximal anatomy of the long (femur) bone (9).

In order for the telescopic plate (8) to move axially in the proximal and distal directions, it includes a D-shaped tip on the proximal and distal region plates (6.1), which passes into the plate central body (4), which is formed to prevent rotation.

1. A telescopic plate (8) used to prevent or stabilize long (femur) bone fractures and correct long (femur) bone deformities in children older than 18 months with osteogenesis imperfecta (glass bone), comprising
the distal region plate (1) and the proximal region plate (6), which form the telescopic plate (8), which can be extended by moving within each other and locked at the desired length,
at least one plate central body (4) in which the distal region plate (1) and the proximal region plate (6) move in a telescopic structure.

2. Telescopic plate (8) according to claim 1, comprising
the distal region plate (1), which is fixed to the distal of the femur bone (9) with the distal region lock screws (2), which are formed so as not to come to the child's growth line.

3. Telescopic plate (8) according to claim 1, comprising
the proximal region plate (6), which is fixed with proximal region lock screws (7), in the proximal of the femur bone (9).

4. Telescopic plate (8) according to claim 1, comprising
plate central body (4) between the distal region plate (1) and the proximal region plates (6),
the plate that allows the telescopic plate (8) to extend in the distal and proximal direction, the plate of the distal region (1) and the plates of the proximal region (6), which slides in the plate central body (4).

5. Telescopic plate (8) according to claim 1, comprising
fixing screws (3) that are tightened with the help of the grooves on the distal and proximal region plates (1, 6) and that prevent the telescopic plate (8) from making any sliding motion by tightening the plate central body (4) after tightening.

6. Telescopic plate (8) according to claim 1, comprising
the plate central body (4), which is fixed to the bone (9) with diaphysis lock screws (5).

7. Telescopic plate (8) according to claim 1, comprising
the structure of the proximal and distal region plates (6.1), which are formed in a way to prevent rotation so that the telescopic plate (8) can move axially in the proximal and distal direction, in the form that passes into the plate central body (4).

8. Telescopic plate (8) according to claim 1, comprising
the plate central body (4) which is designed with a thin profile and suitable for the distal and proximal anatomy of the femur bone (9), fixed with locking and non-locking screws in the distal, proximal and cortical regions on the femur bone (9), and in which the distal region plate (1) and the proximal region plate (6) move in a telescopic structure.

9. Telescopic plate (8) according to claim 1, comprising
the distal region plate (1), the proximal region plate (6), and the plate central body (4) in which stability is achieved with locked and unlocked screws in distal, proximal and cortical regions, and which has a profile structure suitable for the distal and proximal anatomy of the long (femur) bone (9).

10. Telescopic plate (8) according to claim 1, comprising
a D-shaped tip on the proximal and distal region plates (6.1), which passes into the plate central body (4), which is formed to prevent rotation in order for the telescopic plate (8) to move axially in the proximal and distal directions.
